SEQUENCE LISTING (39 sequences)

SEQ ID NO: 1 - protein, 14 aa, Candidatus Competibacter
SAFALSGTNIAALR

SEQ ID NO: 2 - protein, 10 aa, Candidatus Competibacter
FTVVNSGIVR

SEQ ID NO: 3 - protein, 12 aa, Candidatus Competibacter
TEAFIPLVPLDR

SEQ ID NO: 4 - protein, 17 aa, Candidatus Competibacter
GVVTNTLIGVTVADPNR

SEQ ID NO: 5 - protein, 11 aa, Candidatus Competibacter
LVAGTDYTSTR

SEQ ID NO: 6 - protein, 14 aa, Candidatus Competibacter
AGVNTLVGLTTFSK

SEQ ID NO: 7 - protein, 13 aa, Candidatus Competibacter
FYTDSANNEFGGR

SEQ ID NO: 8 - protein, 11 aa, Candidatus Competibacter
ANFGYTGNPLK

SEQ ID NO: 9 - protein, 10 aa, Candidatus Competibacter
TNLYSVFNMT

SEQ ID NO: 10 - protein, 11 aa, Candidatus Accumulibacter
TININPSIAFR

SEQ ID NO: 11 - protein, 11 aa, Candidatus Accumulibacter
TGFWTPAGAAR

SEQ ID NO: 12 - protein, 13 aa, Candidatus Accumulibacter
GLIGSTDLFQWVK

SEQ ID NO: 13 - protein, 15 aa, Candidatus Accumulibacter
TVAVTLLDEAHNPVR

SEQ ID NO: 14 - protein, 11 aa, Candidatus Accumulibacter
TMNINPSIAFK

SEQ ID NO: 15 - protein, 14 aa, Candidatus Accumulibacter
LPGPGTIYMSQSLR

SEQ ID NO: 16 - protein, 13 aa, Candidatus Accumulibacter
FMFGLAYDQSPVK

SEQ ID NO: 17 - protein, 11 aa, Candidatus Accumulibacter
TNPANYHGMLR

SEQ ID NO: 18 - protein, 14 aa, Candidatus Accumulibacter
SAIQYHTEGSASLR

SEQ ID NO: 19 - protein, 15 aa, Candidatus Accumulibacter
YIIGPGDSVNIIVWR

SEQ ID NO: 20 - protein, 16 aa, Candidatus Accumulibacter
FGPVQYSAGATTTKPR

SEQ ID NO: 21 - protein, 9 aa, Propionivibrio
LSWGSATAR

SEQ ID NO: 22 - protein, 12 aa, Propionivibrio
IDSALSAVSSLR

SEQ ID NO: 23 - protein, 12 aa, Propionivibrio
VLEHLPVSALGK

SEQ ID NO: 24 - protein, 11 aa, Propionivibrio
HTEDFGQTLGK

SEQ ID NO: 25 - protein, 14 aa, Propionivibrio
VTTIGDDIAWIKPR

SEQ ID NO: 26 - protein, 11 aa, Propionivibrio
IAFTGSTSTGR

SEQ ID NO: 27 - protein, 12 aa, Propionivibrio
YGVAYDQTPVQR

SEQ ID NO: 28 - protein, 15 aa, Propionivibrio
GVTVNVIAPGYVATK

SEQ ID NO: 29 - protein, 15 aa, Propionivibrio
ALDAVIEAIVDAVAK

SEQ ID NO: 30 - protein, 9 aa, Propionivibrio
ALAAELAAK

SEQ ID NO: 31 - protein, 10 aa, Propionivibrio
INNSQATFGR

SEQ ID NO: 32 - protein, 11 aa, Tabrizicola aquatica
FGIIDSSSAVK

SEQ ID NO: 33 - protein, 9 aa, Tabrizicola aquatica
FIPTTGETR

SEQ ID NO: 34 - protein, 14 aa, Tabrizicola aquatica
FNIDASTETDSGVK

SEQ ID NO: 35 - protein, 12 aa, Tabrizicola aquatica
METDGGVTFGGR

SEQ ID NO: 36 - protein, 9 aa, Tabrizicola aquatica
FGLDNAAFK

SEQ ID NO: 37 - protein, 19 aa, Tabrizicola aquatica
FGLVYNDDGTTSDTNLSYR

SEQ ID NO: 38 - protein, 11 aa, Tabrizicola aquatica
ILGVSGDMGVK

SEQ ID NO: 39 - protein, 13 aa, Tabrizicola aquatica
ALVYGFAGMSSGK

(19) United States
(12) Patent Application Publication
(10) Pub. No.: US 2024/0401102 A1
(43) Pub. Date: Dec. 5, 2024

(54) PROTEIN AND PEPTIDE DATABASE-ENABLED RAPID MONITORING AND QUANTIFICATION OF MICROBES AND ASSOCIATED PRODUCTS

(71) Applicant: Technische Universiteit Delft, Delft (NL)

(72) Inventors: Martin PABST, Delft (NL); Marinus Cornelis Maria VAN LOOSDRECHT, Delft (NL); Hugobert Bernhard Crijn KLEIKAMP, Delft (NL)

(73) Assignee: Technische Universiteit Delft, Delft (NL)

(21) Appl. No.: 18/259,918

(22) PCT Filed: Nov. 29, 2021
(86) PCT No.: PCT/NL2021/050722

§ 371 Date: Jun. 29, 2023

(30) Foreign Application Priority Data
Dec. 31, 2020 (NL) 2027256

(51) Int. Cl. C12Q 1/06; C12Q 1/689; G16B 25/10

(52) U.S. Cl. CPC C12Q 1/06; C12Q 1/689; G16B 25/10

(57) ABSTRACT

The present invention relates to a method of monitoring a microbiome, a method of controlling a reactor comprising a microbiome, or a method of determining an effect of a medicament or drug in an environment comprising a microbiome, wherein in both cases said microbiome is monitored according to said method, and to a microbiome monitoring computer program comprising instructions for monitoring a microbiome, which methods are efficient, relatively quick, and relatively cheap.

### RELATED APPLICATIONS

This application is a national entry of PCT International Patent Application No. PCT/NL2021/050722, filed Nov. 29, 2021, in the name of “TECHNISCHE UNIVERSITEIT DELFT” [NL], which PCT application claims the benefit of priority of Netherlands Patent Application Serial No. 2027256, filed Dec. 31, 2020, in the name of “TECHNISCHE UNIVERSITEIT DELFT” [NL]. The entire contents of the above-referenced applications and of all priority documents referenced in the Application Data Sheet filed herewith are hereby incorporated by reference for all purposes.

### INCORPORATION-BY-REFERENCE OF SEQUENCE LISTING

The present application is being filed along with a Sequence Listing in electronic format. The Sequence Listing is provided as a file entitled Rev_SEQLST-VANP-2023073p-ST25.txt, which is 8,192 bytes in size, created and last modified on Jul. 12, 2024. The information in the accompanying Sequence Listing is incorporated by reference in its entirety into this application.

### FIELD OF THE INVENTION

The present invention relates to a method of monitoring a microbiome, a method of controlling a reactor comprising a microbiome, or a method of determining an effect of a medicament or drug in an environment comprising a microbiome, wherein in both cases said microbiome is monitored according to said method, and to a microbiome monitoring computer program comprising instructions for monitoring a microbiome, which methods are efficient, relatively quick, and relatively cheap.

### BACKGROUND OF THE INVENTION

The present invention is in the field of a method of monitoring a microbiome. A microbiome may be considered to relate to a characteristic microbial community occupying a reasonably well-defined habitat which has distinct physio-chemical properties. The term thus not only refers to the microorganisms involved but typically also encompasses their theatre of activity. The microbiome may be defined as a characteristic microbial community occupying a reasonably well-defined habitat which has distinct physio-chemical properties. The microbiome not only refers to the microorganisms involved but also encompass their theatre of activity, which results in the formation of specific ecological niches. The microbiome, which forms a dynamic and interactive micro-ecosystem prone to change in time and scale, is integrated in macro-ecosystems including eukaryotic hosts, and here crucial for their functioning and health. It is noted that the term “microbiota” is separated from the term microbiome in that the microbiota is considered to consists of the assembly of microorganisms belonging to different kingdoms (Prokaryotes [Bacteria, Archaea], Eukaryotes [e.g., Protozoa, Fungi, and Alga]), while their theatre of activity includes microbial structures, metabolites, mobile genetic elements (such as transposons, phages, and viruses), and relic DNA embedded in the environmental conditions of the habitat (see https://en.wikipedia.org/wiki/Microbiome). The present invention is however more related to the microbial community than to the habitat. The habitat of the microbial community is noted and taken into account, but the present invention relates more to the characterization of the microbial community.

A microbiome is a complex community of microbial species. Not only a large number of different species may occur within one microbiome, but also quantities of the species may vary among species. Often a limited number of microbiome species are dominant in mass, but still such may relate to a significant number of species. In particular in microbial production methods, when for instance food is produced, a more limited number of species is typical present. In that respect attention may be paid to sterilize the habitat of the microbiome before production, therewith inherently limiting a variation within the microbiome. Also the species in the microbiome may vary over time, not only in abundance as in biomass thereof, but also some may become negligible whereas others may become abundant. A similar variation in terms of microbiome strains may occur, adding to the complexity. Even genetic changes may occur. So the characteristics of the microbiome in this respect may vary over time.

It is noted that mixed or enriched microbial cultures are considered promising production systems for biotechnology and pharmaceutical industry. Microbial communities exhibit metabolic capabilities which may be unique or superior compared to pure cultures. Furthermore, many microbial communities live in close relation to humans, or other hosts, and therefore may directly impact on human health and well-being. State of the art measurements of the composition or metabolic potential of microbial communities typically relay on staining, or genetic tools, which are unspecific, or generate very large data, and are very time consuming. Furthermore, the relevant information of the actually expressed biomass/metabolic composition is not obtained thereby, but only information relating to species being potentially present per se.

For obtaining biomass mass protein information spectrometry-based community proteomics (metaproteomics) may be used, which technique measures the proteins directly. Unfortunately, common metaproteomic approaches are also very time consuming, and require very high resolution, expensive instrumentation and advanced bioinformatics tools and knowledge for interpretation, hence are highly complex So currently there are no suitable high throughput approaches which can monitor, and control mixed microbial communities (production systems, or natural consortia) at the biomass/metabolic level (proteins, enzymes) on a routine basis. As a consequence, mixed cultures are commonly used and considered only as a black box, which inherently is difficult to control and to operate. Furthermore, some applications, such as medical applications in relation to the gut microbiome, are hampered since monitoring techniques lack fast and specific methods.

Some scientific articles, rather incidentally, relate to metaproteomics mainly. Mikan et al. in “Metaproteomics reveal that . . . ”, https:/doi.org/10.1038/s41396-019-0503-z, reveal that rapid perturbations in organic matter prioritize functional restructuring over taxonomy in western Arctic Ocean microbiomes during 10 days. They used a novel peptide-based enrichment analysis and observed significant changes (p-value<0.01) in biological and molecular functions associated with carbon and nitrogen recycling. It is noted that (meta) proteomics always uses peptides. The novel enrichment analysis mentioned indicates that they looked for peptide signals that are more abundant in one measurement compared to another one. The method used may be considered as a discovery-based peptide-centric approach. The us of a DNA library is a common procedure in metaproteomics. Here it is used as a database for metaproteomics (in order to obtain amino acid sequences, taxonomy and function). The article is mainly concerned with a data analysis advancement to better get community functions from metaproteomics data. Franzosa et al, in “Sequencing and beyond: integrating molecular ‘omics’ for microbial community profiling”, is a review article mainly concerned with advances in DNA sequencing have enabled culture-independent profiling of microbial community membership and function—the field of metagenomics. These approaches have rapidly expanded our knowledge of human-associated and environmental microbiomes (https:/doi:10.1038/nrmicro3451). Li et al. in “RapidAIM: a culture- and metaproteomics-based Rapid Assay of Individual Microbiome responses to drugs” developed an approach to screen compounds against individual microbiomes in vitro, using metaproteomics to both measure absolute bacterial abundances and to functionally profile the microbiome (doi:10.1186/S40168-020-00806-Z). Calusinska et al. in “A year of monitoring 20 mesophilic full-scale bioreactors reveals the existence of stable but different core microbiomes in bio-waste and wastewater anaerobic digestion systems”, used high-throughput sequencing of small rRNA gene, and a monthly monitoring of the physicochemical parameters for 20 different mesophilic full-scale bioreactors over 1 year, to generate a detailed view of AD microbial ecology towards a better understanding of factors that influence and shape these communities (DOI:10.1186/s13068-018-1195-8).

The present invention therefore relates to a method of monitoring a microbiome and further aspects thereof, which overcomes one or more of the above disadvantages, without compromising functionality and advantages.

### SUMMARY OF THE INVENTION

It is an object of the invention to overcome one or more limitations of the methods of monitoring a microbiome of the prior art and at the very least to provide an alternative thereto. The present invention relates to a new method of monitoring a microbiome, over time, comprising providing the microbiome, the microbiome comprising a population having a population biomass, the population comprising a variety of microbial species and/or a variety of microbial strains (a genetic variant, a subtype or a culture within a biological species) wherein each microbial species and/or microbial strain individually provides a species biomass or strain biomass to the population, wherein microbial species and microbial strains are in particular selected from Archaea, Bacteria, Eukaryote, Algae, Fungi and small protists, (1) within the microbiome characterizing at least 50 wt. % of population biomass in terms of the biological taxonomy, in particular at least 5 levels of taxonomy, and typically 7-9 levels of taxonomy, more in particular the taxonomic levels being selected from Kingdom, Phylum, Class, Order, Family, Genus, Species, and if possible from strain, and sub-strain, microbial species and/or strains being present in the population biomass, in particular characterizing at least 70 wt. % of said population biomass, more in particular characterizing at least 85 wt. % of said population biomass, even more in particular characterizing at least 95 wt. % of said population biomass, (2a) at least two times extracting protein sequences from said population biomass, the two extractions forming a time sequence, which extractions are typically directly made from the biomass, (2d) directly or indirectly determining an amount of at least one extracted protein sequence, wherein the amount may be relative to a total amount or absolute in terms of (bio)mass, and typically said amount is determined using chemical analytics, and (2f comparing in the time sequence a later amount of extracted protein sequence with an earlier amount of protein sequence, such as comparing a last amount of extracted protein sequence with a first amount of extracted protein sequence. In particular the present method relates to a method of monitoring a microbiome which comprises (2c1) selecting a sub-population of the microbial species, wherein said selection is typically done with a computer/dedicated software, (2c2) determining a sub-set of protein sequences and/or peptides representing the sub-population, or metabolic functions of the microbiome, and after step (2d) (2c) directly or indirectly analysing the amounts of extracted protein sequences and/or peptides of the sub-set by comparing the sub-set against a database comprising protein sequences and/or peptides of the sub-set, and determining biomass per microbial species of the sub-population, in particular biomass proteinous contribution per microbial species. In step (2c1) Currently selection is done empirically, e.g. looking for sequences at a selected taxonomic ranking (at least genus), and/or peptides that are good to measure/quantify (physicochemical properties), and/or have uniform proportions, representing the community, or genera of interest. Of course, machine learning may be used in an alternative, in particular once inventors have collected data over many plants, over a longer period of time. Typically a cut-off is used at a lower end of the percentage/amount obtained, such as at 1% or 0.1%. The invention therewith also relates to i) a specifically designed algorithm which extracts system relevant protein/peptide information from metagenomics and proteomics data of the particular enrichment culture or community. The relevant information thereto are proteins/peptides unique for taxonomic rankings, and/or responsible for certain (metabolic) conversions. The invention therewith also relates to ii) thereby generated protein/peptide database containing the system relevant information for a particular stage of the system (e.g. for the core microbiome/function of activated granular sludge water treatment system at different operational stages, gut microbiome at different health stages, or a mixed microbial production system at different productivities). These protein/peptide databases arc found to enable a rapid and quantitative monitoring of mixed microbial cultures, by means of highly simplified instrumentation for measurement (such as low resolution mass spectrometers) and highly simplified software for data analysis. Furthermore, this is found to enable multiplexed sample processing, measurements and high-throughput data processing. Thereby, complete analysis times can easily be reduced to within one working day. With the specifically designed software which extracts information/data from metagenomics and metaproteomics experiments a database with reduced complexity is provided. It is found that the microbial (enrichment) culture peptides/protein database, represents a specific condition of the community/enrichment. The present method provides rapid and focused quantitative monitoring of mixed cultures using e.g. low resolution mass spectrometric approaches. So, by approaching the enormous complexity of microbial consortia and enrichment cultures in a stepwise approach, previously very difficult to monitor and to control systems can now be monitored adequately. Therewith relevant information of the actually expressed biomass/metabolic composition is obtained. It is found that e.g. mass spectrometry based community proteomics (metaproteomics), which measures the proteins directly, overcomes the limitations. Therewith one can monitor, and control/interpret mixed microbial communities (production systems, or natural consortia) at the biomass/metabolic level (proteins, enzymes). The mixed cultures are no longer a black box. Also for other applications, such as medical applications, e.g. in relation to the gut microbiome, are now provided with fast and specific methods. The invention overcomes the above problems, by reducing the complexity to a level, which enables rapid, quantitative and low spec instrumentation/data interpretation monitoring. In case a genome or protein/peptide could not be mapped, it was attributed to a less specific, that is higher taxonomic, level. Typically a genome c.q. peptide/protein sequence could be annotated to more than one close-by member, for a given taxonomic level; then the closest hit was typically chosen, or in an alternative a higher taxonomic level. In the end a majority of possible peptides/protein sequences, which could have been possible on genomic data, were not found in practice, and a more limited set could be used. Moreover, not all peptides are considered suitable for quantitative analysis, therefore peptides can be ranked to not only provide the representative subset of the community, but also the most the most suitable subset from an analytical viewpoint.

In a second aspect the present invention relates to a method of controlling a reactor comprising a microbiome, comprising monitoring a microbiome according to the invention, and based on said microbiome, or changes therein, adapting at least one parameter selected from temperature, flow, pH, static residence time, solid retention time, nitrogen content, phosphorous content, amount of biomass, amount of nutrients, oxygen content, flow, alkalinity content, fatty acids content, redox values, feed flux, production installation of input sludge, method of production of input sludge, age of input sludge, organic carbon content COD of input sludge, method of production of input sludge, dosing of chemicals during production of input sludge, remaining concentration of dosing chemicals left, process setting during production of input sludge, polyelectrolyte concentration, type of polyelectrolyte, bowl speed, pressure applied to the sludge, gas produced, stir rate, ammonium concentration in an effluent stream, concentration of protein sequences, concentration of sugars, concentration of cellulosic material, amount of degradable organic matter, cation concentration, differential speed, trace elements, in particular oxygen content, or not adapting a parameter, or stopping operation of the reactor. The adaptation may be provided as such, or may comprise a feedback loop, in which, based on said microbiome, adaptation is performed.

In a third aspect the present invention relates to a method of determining an effect of a medicament or drug, or of an purposive action, or change of habit, such as when changing a diet, in an environment comprising a microbiome, comprising monitoring a microbiome according to the invention, and adapting an amount of medication or drug, and/or adapting an administration regime of medication or drug, and/or changing purposive action or habit.

In a fourth aspect the present invention relates to a microbiome monitoring computer program comprising instructions for monitoring a microbiome according to the invention or for operating a reactor according to the invention or for determining an effect of a medicament or drug in an environment comprising a microbiome according to the invention, the instructions causing the computer (1) to carry out the following steps: (1) characterizing at least 50% of the microbial species being present in the population, (2d) directly or indirectly determining an amount per extracted protein sequence, and (2f) comparing in the time sequence a later amount of extracted protein sequence with an earlier amount of protein sequence, such as comparing a last amount of extracted protein sequence with a first amount of extracted protein sequence.

In a fifth aspect the present invention relates to a microbiome monitoring computer program comprising instructions for monitoring a microbiome according to the invention or for operating a reactor according to the invention or for determining an effect of a medicament or drug in an environment comprising a microbiome according to the invention, wherein the computer program comprises instructions for learning, such as machine learning, adaptive learning, and combinations thereof.

The present invention provides a solution to one or more of the above mentioned problems and overcomes drawbacks of the prior art.

Advantages of the present description are detailed throughout the description.

### DETAILED DESCRIPTION OF THE INVENTION

In an exemplary embodiment of the present method of monitoring a microbiome characterizing the microbial species in step (1) is a qualitative characterization.

In an exemplary embodiment of the present method of monitoring a microbiome the characterization in step (1) is performed using a genetic sequence of a species and matching said genetic sequence with a genetic sequence-database comprising genetic sequence-data of possible microbial species, such as by genomics or metagenomics, in particular wherein said genetic sequence is a DNA sequence, a RNA sequence, a gene sequence, an enzyme sequence, or part thereof, or combination thereof.

In an exemplary embodiment of the present method of monitoring a microbiome before determining an amount per extracted protein sequence (2b) the extracted protein sequences are cleaved into peptide fragments.

In an exemplary embodiment of the present method of monitoring a microbiome the extracted protein sequences are cleaved into peptide fragments each individually comprising 6-100 amino acids, preferably 7-75 amino acids, in particular 8-55 amino acids, such as 10-12 amino acids.

In an exemplary embodiment of the present method of monitoring a microbiome cleavage is obtained by at least one specific protease, preferably a protease of mixed nucleophilic superfamily A, preferably a serine protease, in particular a chymotrypsin-like protease or a subtilisin-like protease, such as Trypsin (CAS 9002-07-7).

In an exemplary embodiment of the present method of monitoring a microbiome (2d) determining an amount of protein sequence directly is performed using high resolution mass spectrometry or wherein (2d) determining an amount of protein sequence indirectly is performed using high resolution mass spectrometry on peptides and/or proteins.

In an exemplary embodiment of the present method of monitoring a microbiome the step of (1) characterizing at least 50 wt. % of the species being present in the population is performed only once, such as in a well-defined condition, e.g. a reactor performing as expected.

In an exemplary embodiment of the present method of monitoring a microbiome the step of (2c1) selecting a sub-population of the microbial species is performed only once.

In an exemplary embodiment of the present method of monitoring a microbiome the step of (2c2) determining a sub-set of protein sequences representing the sub-population is performed only once.

In an exemplary embodiment of the present method of monitoring a microbiome in step (2c2) protein sequences and/or peptides are selected which represent at least one high taxonomic level, in particular an Order level, a Family level, or Genus level, or represent at least one metabolic pathway present in a variety of species and/or strains, and/or [(Every species can contribute with approximately 2.5K protein sequences and every protein sequence can give approx. 10-50 peptide fragments, so the theoretical numbers get very large)].

In an exemplary embodiment of the present method of monitoring a microbiome in step (2d) determining an amount of at least one extracted protein sequence relates to a relative amount or to an absolute amount. The amount may be determined in weight terms (e.g. mg), but it is found more practical to use a relative amount, or abundance, or likewise peak area in a chromatogram. In a further step these may be compared mutually, or compared to the total protein/peptide signal, or even being relative to an amount of injected peptides, which may be considered to function as a calibration.

In an exemplary embodiment of the present method of monitoring a microbiome a calibration is provided.

In an exemplary embodiment of the present method of monitoring, a protein and/or peptide database of the present microbiome is generated.

In an exemplary embodiment of the present method of monitoring a sub-set of the protein and/or peptide database of the present microbiome is created.

In an exemplary embodiment of the present method of monitoring a microbiome a relative amount is relative to an earlier determined amount or relative to an amount of at least one other protein sequence.

In an exemplary embodiment of the present method of monitoring a microbiome in step (2d) an amount of at least one most abundant extracted protein sequence is determined.

In an exemplary embodiment of the present method of monitoring a microbiome in step (2d) an amount of at least one most characterizing extracted protein sequence is determined, in particular an extracted protein sequence with the most linear quadratic estimate weight.

In an exemplary embodiment of the present method of monitoring a microbiome the steps of (2a) at least two times extracting protein sequences from said population are performed as often as required for process control, such as based on statistical process control, or based on out of range control, or a combination thereof.

In an exemplary embodiment of the present method of monitoring a microbiome in step (1) at least once an amplification technique is used, such as PCR.

In an exemplary embodiment of the present method of monitoring a microbiome the population comprises 10¹-10⁷ different species, in particular 2*10¹-10⁶ different species, more in particular 10²-10⁵ different species.

In an exemplary embodiment of the present method of monitoring a microbiome the sub-population comprises 2-10⁵ of the different species of the population (0.001-10%), in particular 4-10³ of the different species of the population (0.1-1%), more in particular 6-10² different species, such as 7-20 different species.

In an exemplary embodiment of the present method of monitoring a microbiome 10²-10⁸ of different protein sequences are extracted, in particular 10³-10⁴ of different protein sequences.

In an exemplary embodiment of the present method of monitoring a microbiome 10¹-10⁴ of different peptide fragments are formed, in particular 10²-10³ of different peptide fragments.

In an exemplary embodiment of the present method of controlling a reactor comprising a microbiome the reactor is selected from a digestion reactor, a continuous stirred tank reactor, a batch reactor, a repeated batch reactor, a sequence batch reactor, a single reactor with segmented sub-reactors, a plug flow reactor, a post-digestion reactor, a dewatering device, and combinations thereof.

In an exemplary embodiment of the present method of controlling a reactor comprising a microbiome the reactor comprises wastewater, such as wastewater from housings, from industry, from hospitals, from facilities in general, or a food comprising a mixed microbial population, such as bear, wine, a dairy product, such as yoghurt, or cheese, or a fermentation product, or a digestion product, or an enrichment product, or a microbial consortium product.

In an exemplary embodiment of the present method of controlling a reactor comprising a microbiome monitoring is performed 1-168 times per week.

In an exemplary embodiment of the present method of determining an effect of a medicament or drug in an environment comprising a microbiome the microbiome is selected from a mammal, such as a gastro-intestinal microbiome, a skin microbiome, an oral microbiome, a rectal microbiome, a genital tract microbiome, and an urinary microbiome.

In an exemplary embodiment the present microbiome monitoring computer program may further comprise instructions for storing microbiome data, in particular for characterizing a microbial species, a genetic sequence of said microbial species, a protein sequence produced or present of said microbial species, or a peptide fragment produced or present of said microbial species,2

The invention will hereafter be further elucidated through the following examples which are exemplary and explanatory of nature and are not intended to be considered limiting of the invention. To the person skilled in the art it may be clear that many variants, being obvious or not, may be conceivable falling within the scope of protection, defined by the present claims.

### FIGURES

FIGS. 1a,b, 2a,b and 3 show details of the present invention.

### DETAILED DESCRIPTION OF FIGURES

In the figures:

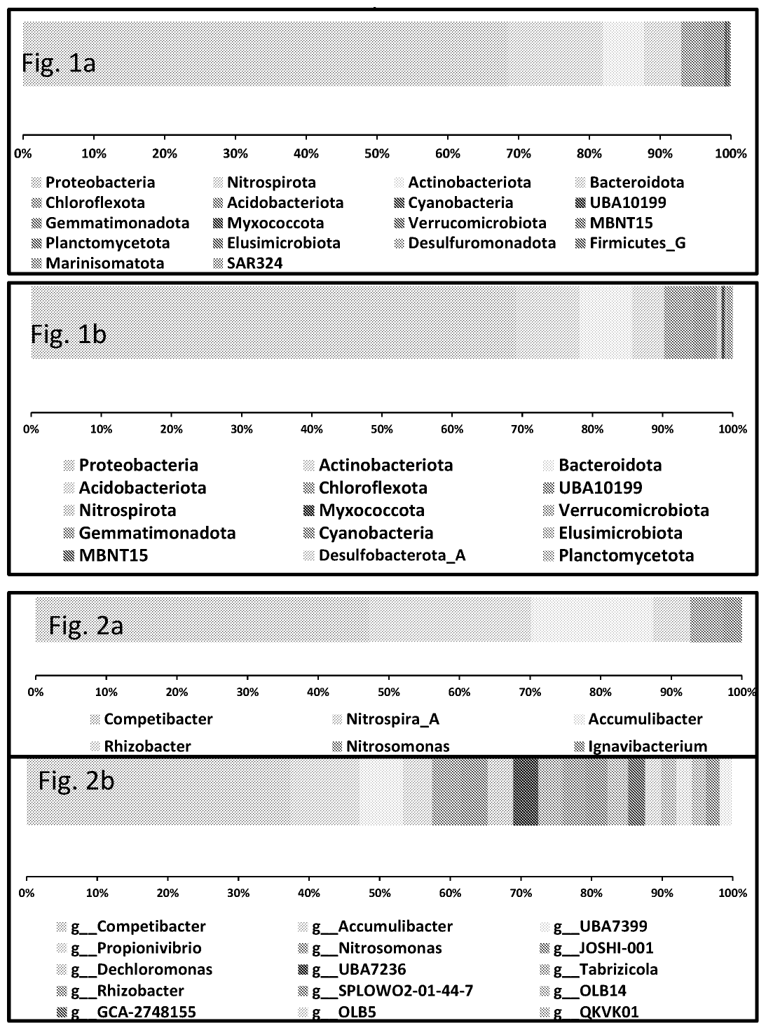

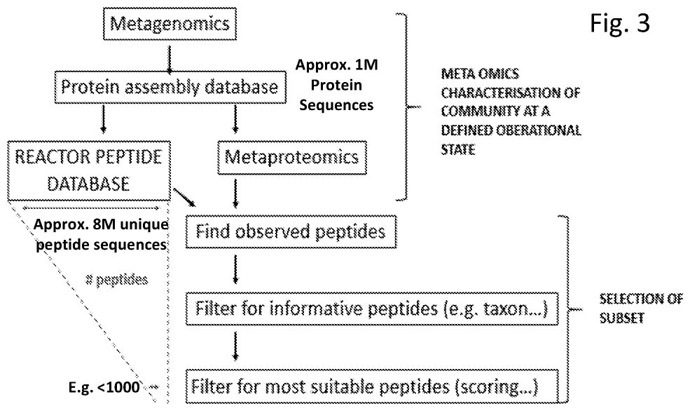

FIG. 1 shows top phylum levels for WWTP-I (FIG. 1b) and WWTP-II (FIG. 1a) as established from the granules by metaproteomics. The microbiomes are comparable, which may be expected in view of both examples relating to a WWTP, and at the same time results show clear differences in abundances. These differences may be attributed to different performances of the respective plants considering their different location, wastewater and possibly different operation.

FIG. 2 shows top 75% genera compromising 75% of peptide peak areas for WWTP-I (FIG. 2b) and WWTP-II (FIG. 2a) as established from the granules by metaproteomics. The microbiomes are again comparable but again show clear differences in the abundance of individual members. These differences may be attributed to different performances of the respective plants considering their different location, wastewater and possibly different operation.

FIG. 3 shows the general process for the peptide subset selection procedure used for routine monitoring of the microbial community. Given numbers are based on the example from the WWTP-I. Initial metagenomics and metaproteomics experiments identify the (commonly) observed peptide sequences, which are further narrowed down by selecting for taxon informative peptides (e.g. genus or species level) or metabolic function informative peptides. The informative peptides are further filtered for the methodologically most suitable sequences, equally representing the microbiome, or specific community members.

### EXPERIMENTS

First of all a number of operating reactors were identified. On series of reactors relate to so-called Nereda reactors These reactors produce bacterial sludge. Aerobic granular sludge and anammox granular sludge, and the processes used for obtaining them are known to a person skilled in the art. For the uninitiated, reference is made to Water Research, 2007, doi: 10.1016/j.watres.2007.03.044 (anammox granular sludge) and Water Science and Technology, 2007, 55(8-9), 75-81 (aerobic granular sludge), as well as previous applications in this respect from the same applicant. From these reactors microbiomes were retrieved. These microbiomes were characterized and monitored according to the invention. Metadata or raw data derived from these Nereda plants relates to:
1. metagenomics data from 3 Nereda waste water treatment plants;
2. conventional metaproteomics data from the same 3 Nerada plants;
3. a peptide database which contains all possible peptides (exemplified from one Nereda plant);
4. a peptide database which contains a subset of observed peptides (exemplified for one Nereda plant);
5. a peptide database which contains a selected subset used for further monitoring (from the database described directly above)

In order to monitor these plants over time, Nereda treatment plant samples, from different locations, were obtained. Including those which are found to operate sub-optimal. The 2 plants look comparable in regards to the microbiome. It is considered that these similarity is understood that the core functions operate in a similar way.

The following section relates to raw data from a to be submitted scientific publication of H. B. C. Kleikamp et al, entitled “A deep comparative metaproteomic investigation of the core microbiome of aerobic granular sludge”, which publication and its contents are incorporated by reference.

Here inventors compare metagenomics and metaproteomics based microbiome analysis for three full-scale aerobic granular sludge wastewater treatment plants (Nereda™ technology). To enable rapid metaproteomic sampling at reduced cost, further improvements are made to the existing proteomics pipeline by using habitat specific databases and the investigation for taxon and metabolic function relevant peptides. Differences in observed taxonomic and functional distributions for the successfully operating Dutch AGS plants using 16S, metagenomic and metaproteomic analysis is discussed. This gives a core microbiome of aerobic granular sludge systems. The evaluation shows that proteomics is more suitable than genomics for the biomass characterisation of the plants.

### Experiments

### Sampling, Protein Extraction and Proteolytic Digestion

Activated granular sludge was sampled from two of the above waste water treatment plants (WWTP). Granules (approx. 2.0 mm diameter) were freeze dried and grinded using a mortar and pestle and further subjected to beads beating using glass beads in a TEAB/B-PER buffer. Following an additional freeze/thaw step and incubation at elevated temperature (95° C.) for a short time period, the tubes were cooled and centrifuged at full speed using a bench top centrifuge for 10 minutes. The supernatant was collected and protein was precipitated using trichloroacetic acid (TCA). Following a short cooling the solution was centrifuged at full speed using a bench top centrifuge to collect the protein pellet. The protein pellet was washed once with ice cold acetone and reconstituted in 6M Urea (aiming for a protein concentration of 1 μg/μL), reduced using Dithiothreitol (DTT) and alkylated using Iodoacetamide (IAA). The protein solution was finally diluted to below IM urea using 200 mM bicarbonate buffer, before addition of sequencing grade trypsin at a trypsin: protein ratio of approx. 1:50. Samples were digested at 37° C. over-night. Obtained peptides were desalted using an Oasis HLB SPE well plate (Waters), according to the manufacturers protocol. The eluate was speed vacuum dried and resolubilised in 0.1% TFA solution for further prefractionation using a high pH reverse phase peptide fractionation kit (Thermo) according to the protocol provided by the manufacturer. Fractions were speed-vacuum dried and resolubilised in H₂O, containing 0.1% formic acid and 3% acetonitrile. Peptide/protein contents were estimated using a Nanodrop spectrophotometer.

### Shotgun Metaproteomic Analysis

Aliquots of the fractions, corresponding to approx. 250 ng protein, were analysed in duplicates using a shot-gun proteomics approach. Briefly, the samples were analysed using a nano-liquid-chromatography system consisting of an ESAY nano LC 1200, equipped with an Acclaim PepMap RSLC RP C18 separation column (50 μm×150 mm, 2 μm and 100 A), and a QE plus Orbitrap mass spectrometer (Thermo). The flow rate was maintained at 300 nL/min over a linear gradient to 30% solvent B over 60 or 90 minutes, and finally to 75% B over additional 30 minutes. Solvent A consisted of H₂O containing 0.1% formic acid, and solvent B consisted of 80% acetonitrile in H₂O and 0.1% formic acid. The Orbitrap was operated in data-dependent acquisition (DDA) mode where the top 10 mass peaks were isolated and fragmented using a NCE of 28. The AGC target was set to 1e5, at a max IT of 54 ms and 17.5K resolution at MS2.

### DNA Extraction and Metagenomic Analysis

DNA from granules was extracted using the DNeasy UltraClean Microbial Kit (Qiagen, The Netherlands). Following extraction, DNA was checked for quality by gel electrophorese and by using a Qubit 4 Fluorometer (Thermo Fisher Scientific, USA). Metagenomic sequencing was performed by Novogene Ltd. (Hongkong, China). Briefly, for library construction, a total amount of 1 μg DNA per sample was used as input material. Sequencing libraries were generated using NEBNext® Ultra™ DNA Library Prep Kit for Illumina (NEB, USA) following manufacturer's recommendations. The DNA sample was fragmented by sonication to a size of 350 bp, then DNA fragments were end-polished, A-tailed, and ligated with the full-length adaptor for Illumina sequencing with further PCR amplification. PCR products were purified and libraries were analysed for their size distribution using an Agilent 2100 Bioanalyzer, and quantified using real-time PCR. The clustering of the index-coded samples was performed on a cBot Cluster Generation System according to the manufacturer's instructions. After cluster generation, the library preparations were sequenced on an Illumina HiSeq platform and paired-end reads were generated. Raw reads were quality checked and statistical low-quality reads were trimmed. Trimmed reads were assembled using metaSPAdes v3.13.0 with default settings. For scaffolds larger than 1500 base pairs, taxonomic affiliation was determined using RefineM. Taxonomic annotation was performed according to GTDB.

### Metaproteomic Data Analysis and Peptide Sequence Database Processing

The mass spectrometric raw data were analysed using the bioinformatics software solution PEAKS Studio X using the metagenomics constructed protein assembly database obtained from the (AGS) granule material. Data were analysed allowing for 20 ppm parent ion and 0.02 m/z fragment ion mass error, 2 missed cleavages, carbamido methylation as fixed and methionine oxidation and N/Q deamidation as variable modifications. Peptide spectrum matches were filtered against 1% false discovery rate (FDR) and protein identifications with≥2 unique peptides were accepted as significant. The identified peptide sequences were further matched against a peptide database unique for the present activated granular sludge microbiome—constructed from the metagenomics database—using Matlab R2020b, to assign a taxonomic lineage to the obtained peptide sequences using the lowest common ancestor approach (LCA). Moreover, sequences were accessed for common occurrence between treatment plants and suitability for quantification, e.g. by considering such as the presence of chemical modifications, abundance or additional scoring parameters. Finally, a subset of the genus level peptides was selected to uniformly represent the community or to represent specified taxa, or functions of interest. Taxon proportions are represented here by summing up (unique) peptide sequence frequencies, or their intensities respectively.

### RESULTS

Two waste water treatment plants (WWTP-I and WWTP-II, located in the Netherlands, were analysed. Table 1 shows metagenomics results annotations of top taxonomic levels of granules of WWTP-I

Table 1: shows the top identified taxonomies by metagenomics (based on % mapped reads) from granules of the WWTP-I, detailed from the Phylum to Genus level. Data are cut-off at a certain level, so not all data is shown

FIGS. 1a-b show a graphical representation of metaproteomics data obtained from WWTP-II (FIG. 1a) and WWTP-I (FIG. 1b). From a somewhat distinct perspective the two data-sets are rather comparable, and clearly have some differences.

TABLE 1

| | % of mapped reads |
|---|---|
| Phylum | |
| Proteobacteria | 19.61% |
| Bacteroidota | 14.06% |
| Acidobacteriota | 4.31% |
| Actinobacteriota | 3.56% |
| Chloroflexota | 1.85% |
| Gemmatimonadota | 0.95% |
| Myxococcota | 0.91% |
| Verrucomicrobiota | 0.44% |
| unclassified | 0.39% |
| Nitrospira | 0.32% |
| Class | |
| Gammaproteobacteria | 14.36% |
| Bacteroidia | 12.73% |
| Alphaproteobacteria | 5.22% |
| Actinobacteria | 3.22% |
| Vicinamibacteria | 1.89% |
| Anaerolineae | 1.64% |
| Ignavibacteria | 1.23% |
| Thermoanaerobaculia | 1.08% |
| unclassified | 0.97% |
| Gemmatimonadetes | 0.93% |
| Polyangia | 0.83% |
| Acidobacteriae | 0.71% |
| Verrucomicrobiae | 0.44% |
| Holophagae | 0.32% |
| Nitrospiria | 0.32% |
| Acidimicrobiia | 0.31% |
| Family | |
| Burkholderiaceae | 6.14% |
| Chitinophagaceae | 4.33% |
| Rhodocyclaceae | 4.32% |
| unclassified | 3.51% |
| Saprospiraceae | 2.42% |
| Dermatophilaceae | 2.40% |
| BACL12 | 2.37% |
| Rhodobacteraceae | 2.04% |
| UBA2999 | 1.66% |
| Sphingomonadaceae | 1.50% |
| envOPS12 | 1.27% |
| PHOS-HE28 | 1.16% |
| Gemmatimonadaceae | 0.88% |
| UBA5704 | 0.84% |
| Competibacteraceae | 0.84% |
| Haliangiaceae | 0.70% |
| Bryobacteraceae | 0.65% |
| OLB5 | 0.59% |
| Ignavibacteriaceae | 0.56% |
| Gallionellaceae | 0.54% |
| Steroidobacteraceae | 0.43% |
| Pedosphaeraceae | 0.35% |
| Holophagaceae | 0.32% |
| Nitrospiraceae | 0.32% |
| Anderseniellaceae | 0.30% |
| Order | |
| Burkholderiales | 12.01% |
| Chitinophagales | 9.59% |
| Actinomycetales | 2.67% |
| Rhodobacterales | 2.05% |
| Vicinamibacterales | 1.85% |
| unclassified | 1.78% |
| Sphingomonadales | 1.51% |
| Flavobacteriales | 1.50% |
| Anaerolineales | 1.35% |
| Rhizobiales | 1.07% |
| Gemmatimonadales | 0.93% |
| UBA5704 | 0.85% |
| Competibacterales | 0.84% |
| Bacteroidales | 0.78% |
| Haliangiales | 0.70% |
| Bryobacterales | 0.66% |
| SJA-28 | 0.62% |
| Ignavibacteriales | 0.60% |
| Steroidobacterales | 0.44% |
| Pedosphaerales | 0.40% |
| Propionibacteriales | 0.36% |
| Cytophagales | 0.34% |
| Caulobacterales | 0.33% |
| Holophagales | 0.32% |
| Nitrospirales | 0.32% |
| Genus | |
| unclassified | 8.81% |
| UBA7236 | 2.04% |
| Ferruginibacter | 1.73% |
| JOSHI-001 | 1.68% |
| JJ008 | 1.53% |
| PHOS-HE28 | 1.15% |
| Propionivibrio | 1.05% |
| Tabrizicola | 1.04% |
| GCA-2748155 | 1.00% |
| OLB14 | 0.94% |
| Rubrivivax | 0.91% |
| Dechloromonas | 0.79% |
| Competibacter | 0.78% |
| UBA5704 | 0.77% |
| Accumulibacter | 0.74% |
| SCN-70-22 | 0.73% |
| UBA2376 | 0.64% |
| Rhodoferax | 0.63% |
| OLB5 | 0.59% |
| Sulfuritalea | 0.55% |
| Fen-999 | 0.55% |
| UBA690 | 0.54% |
| Ignavibacterium | 0.53% |
| Ga0077559 | 0.50% |
| Rhizobacter | 0.44% |

Table 2a shows top phylum levels for WWTP-I,

Table 2b shows top phylum levels for WWTP-II

| 2a. Taxon | | | 2b. Taxon | | |
|---|---|---|---|---|---|
| (Phylum level) | | | hylum level) | | |
| WWTP-II | Total intensity | % | WWTP-I | Total intensity | % |
| Proteobacteria | 52754122670 | 69.2 | Proteobacteria | 25408237260 | 68.6 |
| Actinobacteriota | 6816455200 | 8.9 | Nitrospirota | 4913487100 | 13.27 |
| Bacteroidota | 5715209590 | 7.5 | Actinobacteriota | 2172902210 | 5.87 |
| Acidobacteriota | 3454495810 | 4.5 | Bacteroidota | 1937695600 | 5.23 |
| Chloroflexota | 3116359250 | 4.1 | Chloroflexota | 1177024000 | 3.18 |
| UBA10199 | 2566575090 | 3.4 | Acidobacteriota | 1109375100 | 3 |
| Nitrospirota | 541768900 | 0.7 | Cyanobacteria | 68673000 | 0.19 |
| Myxococcota | 381402200 | 0.5 | UBA10199 | 63995000 | 0.17 |
| Verrucomicrobiota | 261479400 | 0.3 | Gemmatimonadota | 55265900 | 0.15 |
| Gemmatimonadota | 209259400 | 0.3 | Myxococcota | 30523600 | 0.08 |
| Cyanobacteria | 164637200 | 0.2 | Verrucomicrobiota | 21798100 | 0.06 |
| Elusimicrobiota | 97227400 | 0.1 | MBNT15 | 17971800 | 0.05 |
| MBNT15 | 39840000 | 0.1 | Planctomycetota | 15434900 | 0.04 |
| Desulfobacterota_A | 38363100 | 0.1 | Elusimicrobiota | 14464000 | 0.04 |
| Planctomycetota | 10307700 | <0.1 | Desulfuromonadota | 9993000 | 0.03 |
| Firmicutes_G | 6577000 | <0.1 | Firmicutes_G | 8436500 | 0.02 |
| Poribacteria | 4628000 | <0.1 | Marinisomatota | 7692000 | 0.02 |
| Desulfuromonadota | 1917800 | <0.1 | SAR324 | 5080200 | 0.01 |

Tables 2a,b show the underlying data for the metaproteomics established microbiome composition bar graphs shown in FIG. 1a,b. Again results are quite comparable, which may be expected in view of both examples relating to a WWTP, and at the same time results show clear difference. These differences may be attributed to different performances of the respective plants.

FIGS. 2a and 2b represent a graphical display relating to genera compromising 75% of peptide peak areas.

Tables 3a (WWTP-II) and 3b (WWTP-I) show the underlying data.

Tables 3a,b show the underlying data for the metaproteomics established microbiome composition bar graphs shown in FIGS. 2a,b.

| 3a. Taxon | | | 3b. Taxon | | |
|---|---|---|---|---|---|
| (Genus level) | Total | | (Genus level) | Total | |
| WWTP-I | intensity | % | WWTP-II | intensity | % |
| Competibacter | 10200821350 | 33.3 | Competibacter | 15044194910 | 26.2 |
| Nitrospira_A | 4913487100 | 16 | Accumulibacter | 3944569200 | 6.9 |
| Accumulibacter | 3732808230 | 12.2 | UBA7399 | 2450375500 | 4.3 |
| Rhizobacter | 1133889810 | 3.7 | Propionivibrio | 1682610290 | 2.9 |
| Nitrosomonas | 1010608500 | 3.3 | Nitrosomonas | 1622785500 | 2.8 |
| Ignavibacterium | 564430700 | 1.8 | JOSHI-001 | 1531125500 | 2.7 |
| Fen-999 | 541494000 | 1.8 | Dechloromonas | 1467696200 | 2.6 |
| Dechloromonas | 516698100 | 1.7 | UBA7236 | 1447233250 | 2.5 |
| Microbacterium | 478092200 | 1.6 | Tabrizicola | 1356120100 | 2.4 |
| JOSHI-001 | 476970200 | 1.6 | Rhizobacter | 1312651900 | 2.3 |
| OLB14 | 375720300 | 1.2 | SPLOWO2-01-44-7 | 1237316400 | 2.2 |
| GCA-2699125 | 349522000 | 1.1 | OLB14 | 1207645300 | 2.1 |
| Propionivibrio | 346273900 | 1.1 | GCA-2748155 | 946845400 | 1.6 |
| Ga0077559 | 317533100 | 1 | OLB5 | 928050900 | 1.6 |
| UBA690 | 308181500 | 1 | QKVK01 | 859242600 | 1.5 |
| | | | Bowdeniella | 856436900 | 1.5 |
| | | | Methylomicrobium | 818980500 | 1.4 |
| | | | Ga0077526 | 775912200 | 1.4 |
| | | | Rubrivivax | 750710610 | 1.3 |
| | | | Acidovorax_B | 679174800 | 1.2 |
| | | | Pedococcus | 624438000 | 1.1 |
| | | | UBA5704 | 595463600 | 1 |
| | | | ZC4RG36 | 580422500 | 1 |
| | | | 39-52-133 | 547569800 | 1 |

FIG. 3 shows schematics of reducing data for monitoring. Starting with some 850000 protein sequences from metagenomics, which would result in some 7.7 million peptide sequences (considering the selected constraints) a reduced set of to be monitored peptides of some 650 peptides is selected (see also table 4).

TABLE 4

| .WWTP-I | # peptides |
|---|---|
| Theoretically possible peptides by metagenomics | 7738222 |
| Actually observed peptides | 23529 |
| Total specific to genus level (and lower) | 8436 |
| Selected subset genus (and lower) | 655 |

Table 5 shows examples of peptide sequences for WWTP-I to be monitored. These sequences are for clarification and need not be searched.

| Competibacter | | | |
|---|---|---|---|
| (Genus level) | Accumulibacter | Propionivibrio | Tabrizicola |
| SEQ ID No. 1 | SEQ ID No. 10 | SEQ ID No. 21 | SEQ ID No. 32 |
| SAFALSGTNIAALR | TININPSIAFR | LSWGSATAR | FGIIDSSSAVK |
| SEQ ID No. 2 | SEQ ID No. 11 | SEQ ID No. 22 | SEQ ID No. 33 |
| FTVVNSGIVR | TGFWTPAGAAR | IDSALSAVSSLR | FIPTTGETR |
| SEQ ID No. 3 | SEQ ID No. 12 | SEQ ID No. 23 | SEQ ID No. 34 |
| TEAFIPLVPLVDR | GLIGSTDLFQWVK | VLEHLPVSALGK | FNIDASTETDSGVK |
| SEQ ID No. 4 | SEQ ID No. 13 | SEQ ID No. 24 | SEQ ID No. 35 |
| GVVTNTLIGVTVADPNR | TVAVTLLDEAHNPVR | HTEDFGQTLGK | METDGGVTFGGR |
| SEQ ID No. 5 | SEQ ID No. 14 | SEQ ID No. 25 | SEQ ID No. 36 |
| LVAGTDYTSTR | TMNINPSIAFK | VTTIGDDIAWIKPR | FGLDNAAFK |
| SEQ ID No. 6 | SEQ ID No. 15 | SEQ ID No. 26 | SEQ ID No. 37 |
| AGVNTLVGLTTFSK | LPGPGTIYMSQSLR | IAFTGSTSTGR | FGLVYNDDGTTSD |
| | | | TNLSYR |
| SEQ ID No. 7 | SEQ ID No. 16 | SEQ ID No. 27 | SEQ ID No. 38 |
| FYTDSANNEFGGR | FMFGLAYDQSPVK | YGVAYDQTPVQR | ILGVSGDMGVK |
| SEQ ID No. 8 | SEQ ID No. 17 | SEQ ID No. 28 | SEQ ID No. 39 |
| ANFGYTGNPLK | TNPANYHGMLR | GVTVNVIAPGYVATK | ALVYGFAGMSSGK |
| SEQ ID No. 9 | SEQ ID No. 18 | SEQ ID No. 29 | |
| TNLYSVFNMT | SAIQYHTEGSASLR | ALDAVIEAIVDAVAK | ..... |
| .... | SEQ ID No. 19 | SEQ ID No. 30 | |
| | YIIGPGDSVNIIVWR | ALAAELAAK | |
| | SEQ ID No. 20 | SEQ ID No. 31 | |
| | FGPVQYSAGATTTKPR | INNSQATFGR | |
| | ..... | ..... | |

The sequences are examples of the selected peptide sequences which would be used to specifically monitor Competibacter, as well as others. for the other genera found present in the treatment plants.

1. A method of monitoring a microbiome comprising
providing the microbiome, the microbiome comprising a population having a population biomass, the population comprising at least one of a variety of microbial species and a variety of microbial strains wherein at least one of each microbial species and/or microbial strain individually provides at least one of a species biomass and strain biomass to the population, wherein microbial species and microbial strains are in particular selected from Archaea, Bacteria, Eukaryote, Algae, Fungi, and small protists,
(1) within the microbiome characterizing at least 50 wt. % of population biomass in terms of biological taxonomy, in particular of the at least one microbial species and/or microbial strains, being present in the population biomass, in particular characterizing at least 70 wt. % of said population biomass, more in particular characterizing at least 85 wt. % of said population biomass, even more in particular characterizing at least 95 wt. % of said population biomass,
(2a) at least two times extracting protein sequences from said population biomass, the two extractions forming a time sequence,
(2c1) selecting a sub-population of the microbial species,
(2c2) determining a sub-set of at least one of protein sequences and peptides representing at least one of the sub-population, and metabolic functions of the microbiome,
(2d) at least one of directly and indirectly determining an amount of at least one extracted protein sequence,
(2e) at least one of directly and indirectly analysing the amounts of extracted at least one of protein sequences and peptides of the sub-set by comparing the sub-set against a database comprising at least one of protein sequences and peptides of the sub-set, and determining biomass per microbial species of the sub-population, and
(2f) comparing in the time sequence a later amount of extracted protein sequence with an earlier amount of protein sequence.

2. The method of monitoring a microbiome according to claim 1, wherein characterizing the microbial species in step (1) is a qualitative characterization.

3. The method of monitoring a microbiome according to any of claim 2, wherein the characterization in step (1) is performed using a genetic sequence of a species and matching said genetic sequence with a genetic sequence-database comprising genetic sequence-data of possible microbial species, in particular wherein said genetic sequence is selected from a DNA sequence, a RNA sequence, a gene sequence, an enzyme sequence, a part thereof, and a combination thereof.

4. The method of monitoring a microbiome according to claim 1, wherein before (2d) determining an amount per extracted protein sequence
(2b) the extracted protein sequences of step (2a) are cleaved into peptide fragments.

5. The method of monitoring a microbiome according to claim 4, wherein the extracted protein sequences of step (2a) are cleaved into peptide fragments each individually comprising 6-100 amino acids.

6. The method of monitoring a microbiome according to claim 4, wherein cleavage is obtained by at least one specific protease.

7. The method of monitoring a microbiome according to claim 5, wherein (2d) determining an amount of protein sequence directly is performed using high resolution mass spectrometry, and wherein (2d) determining an amount of protein sequence indirectly is performed using high resolution mass spectrometry on at least one of peptides and proteins.

8. The method of monitoring a microbiome according to claim 1, wherein the step of (1) characterizing at least 50 wt. % of the species being present in the population is performed only once, and
wherein the step of (2c1) selecting a sub-population of the microbial species is performed only once, and/or
wherein the step of (2c2) determining a sub-set of protein sequences representing the sub-population is performed only once, and
wherein in step (2c2) at least one of protein sequences and peptides are selected which represent at least one characteristic selected from a high taxonomic level, in particular selected from an Order level, a Family level, and a Genus level, a metabolic pathway present in at least one variety of species and strains, and
in step (2d) determining an amount of at least one extracted protein sequence relates to at least one of a relative amount and an absolute amount, and
wherein a relative amount is relative to one of an earlier determined amount and relative to an amount of at least one other protein sequence, and
wherein in step (2d) an amount of at least one most abundant extracted protein sequence is determined, and
wherein in step (2d) an amount of at least one most characterizing extracted protein sequence is determined, in particular an extracted protein sequence with the most linear quadratic estimate weight.

9. The method of monitoring a microbiome according to claim 1,
wherein the steps of (2a) at least two times extracting protein sequences from said population are performed as often as required for process control, and
wherein in step (1) at least once an amplification technique is used, and
wherein the population comprises 10¹-10⁷ different species, in particular 2*10¹-10⁶ different species, more in particular 10²-10⁵ different species, and/or
wherein the sub-population comprises 2-10⁵ of the different species of the population (0.001-10%), in particular 4-10³ of the different species of the population (0.1-1%), more in particular 6-10² different species, such as 7-20 different species, and
wherein 10²-10⁸ of different protein sequences are extracted, in particular 10³-10⁴ of different protein sequences, and
wherein 10¹-10⁴ of different peptide fragments are formed, in particular 10²-10³ of different peptide fragments, and
wherein a calibration is provided, and
wherein a protein and/or peptide database of the present microbiome is generated, and/or
wherein a sub-set of the protein and/or peptide database of the present microbiome is created.

10. The method of controlling a reactor comprising a microbiome, comprising
monitoring a microbiome according to claim 1, and
one of adapting at least one parameter selected from temperature, flow, pH, static residence time, solid retention time, nitrogen content, phosphorous content, amount of biomass, amount of nutrients, oxygen content, flow, alkalinity content, fatty acids content, redox values, feed flux, production installation of input sludge, method of production of input sludge, age of input sludge, organic carbon content COD of input sludge, method of production of input sludge, dosing of chemicals during production of input sludge, remaining concentration of dosing chemicals left, process setting during production of input sludge, polyelectrolyte concentration, type of polyelectrolyte, bowl speed, pressure applied to the sludge, gas produced, stir rate, ammonium concentration in an effluent stream, concentration of protein sequences, concentration of sugars, concentration of cellulosic material, amount of degradable organic matter, cation concentration, differential speed, trace elements, in particular oxygen content, or and not adapting a parameter, and
stopping operation of the reactor.

11. The method according to claim 10, wherein the reactor is selected from a digestion reactor, a continuous stirred tank reactor, a batch reactor, a repeated batch reactor, a sequence batch reactor, a single reactor with segmented sub-reactors, a plug flow reactor, a post-digestion reactor, a dewatering device, and combinations thereof.

12. The method according to claim 10, wherein the reactor comprises a material selected from wastewater, a food comprising a mixed microbial population, wine, a dairy product, a fermentation product, a digestion product, of an enrichment product, and a microbial consortium product.

13. The method according to claim 10, wherein monitoring is performed 1-168 times per week.

14. The method of determining an effect of at least one of a medicament, a drug, an purposive action, or a change of habit, in an environment comprising a microbiome, comprising
monitoring a microbiome according to claim 1, and
adapting at least one of an amount of medication and an amount of drug, and
adapting an administration regime of at least one of medication and drug, and
changing at least one of purposive action and habit.

15. The method according to claim 14, wherein the microbiome is selected from a mammal.

16. A microbiome monitoring computer program comprising instructions for at least one of monitoring a microbiome according to claim 1, and for operating a reactor according to claim 10, and for determining an effect of at least one of a medicament and drug in an environment comprising a microbiome according to claim 14, the instructions causing the computer to carry out the following steps:
(1) within the microbiome characterizing at least 50% of the microbial species being present in the population,
(2d) at least one of directly and indirectly determining an amount per extracted protein sequence, and
(2f) comparing in the time sequence a later amount of extracted protein sequence with an earlier amount of protein sequence, in particular by comparing a last amount of extracted protein sequence with a first amount of extracted protein sequence.

17. The microbiome monitoring computer program according to claim 16, further comprising instructions for storing microbiome data, in particular for characterizing a microbial species, a genetic sequence of said microbial species, a protein sequence produced or present of said microbial species, and at least one of a peptide fragment produced and present of said microbial species.

18. A microbiome monitoring computer program comprising instructions for at least one of monitoring a microbiome according to claim 1, and for operating a reactor according to claim 10, and for determining an effect of at least one of a medicament and drug in an environment comprising a microbiome according to claim 14, wherein the computer program comprises instructions for learning selected from machine learning, adaptive learning, and combinations thereof.
